(19) United States
(12) Patent Application Publication
(10) Pub. No.: US 2016/0214948 A1
(43) Pub. Date: Jul. 28, 2016

(54) PRODRUGS OF MONOMETHYL FUMARATE (MMF)

(71) Applicant: RATIOPHARM GMBH, Ulm (DE)

(72) Inventors: Wolfgang ALBRECHT, Ulm (DE); Roland SELIG, Ulm (DE); Sebastian RABE, Neu-Ulm (DE); Richard GUSERLE, Kötz (DE); Annemarie MAIER, Biberach (DE)

(21) Appl. No.: 15/023,370

(22) PCT Filed: Dec. 20, 2013
(86) PCT No.: PCT/EP2013/077668

§ 371 Date: Mar. 19, 2016

(30) Foreign Application Priority Data
Sep. 27, 2013 (EP) 13186500.8
Dec. 5, 2013 (EP) 13005671.6

(51) Int. Cl. C07D 295/088; C07C 69/78

(52) U.S. Cl. CPC C07D 295/088; C07C 69/78

(57) ABSTRACT

The present invention relates to novel compounds for use as a medicament. In particular, the present invention relates to a medicament, preferably in the treatment and/or prevention of systemic diseases, autoimmune diseases, inflammatory diseases, for example multiple sclerosis and psoriasis.

The present invention relates to novel compounds for use as a medicament. In particular, the present invention relates to novel prodrugs of monomethyl fumarate (MMF) suitable as a medicament, preferably in the treatment and/or prevention of systemic diseases, autoimmune diseases, inflammatory diseases, for example multiple sclerosis and psoriasis. Further, the invention relates to a pharmaceutical composition comprising the novel compounds.

### BACKGROUND OF THE INVENTION

Dimethyl fumarate (DMF) is an oral therapeutic agent which is reported to reduce the rejection often occurring in connection with organ transplantation (host versus graft reaction). Further, DMF is approved to be suitable as medicament for the treatment or prevention of a variety of diseases. For example, DMF is proposed in the treatment of autoimmune diseases such as multiple sclerosis. Further, DMF is suggested to be a suitable active pharmaceutical agent in the treatment of psoriasis.

DMF is characterized by the following chemical Formula (1):

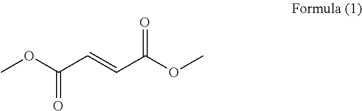

When taken orally DMF is reported to be hydrolyzed for example by the acidic ambience of the stomach or by esterases in the intestine to monomethyl fumarate (MMF), which can be regarded as a metabolite of DMF and can be characterized by the following chemical Formula (2):

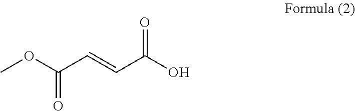

The mechanisms of action of DMF or its metabolite MMF is reported to include inhibition of cytokine-induced nuclear translocation of the nuclear factor kappa B (NF-κB), apoptosis of stimulated T cells, and increased production of the Tₕ2 cytokines IL-4 and IL-5 in stimulated T cells, whereas generation of the Tₕ1 cytokine interferon gamma (IFN-γ) is supposed to remain unaffected. DMF is described to activate the transcription factor Nrf2 (nuclear factor erythroid 2-related factor 2), which binds to antioxidant response elements in the promoters of protective genes such as NADPH-quinone-oxidoreductase-1 (NQO1) and heme-oxygenase-1. Thus, this ultimately raises the levels of the important intracellular antioxidant glutathione (cf. Albrecht P. et al., Journal of Neuroinflammation 2012, 9:163).

Further, it is alleged that the treatment of animals or primary cultures of CNS cells with DMF or MMF resulted in increased nuclear levels of active Nrf2, with subsequent up-regulation of canonical antioxidant target genes. DMF or MMF treatment increased cellular redox potential, glutathione, ATP levels, and mitochondrial membrane potential in a concentration-dependent manner. Treating astrocytes or neurons with DMF or MMF also significantly improved cell viability after toxic oxidative challenge in a concentration-dependent manner. This effect on viability was lost in cells that had eliminated or reduced Nrf2. These data suggest that DMF and MMF are cytoprotective for neurons and astrocytes against oxidative stress-induced cellular injury and loss, potentially via up-regulation of an Nrf2-dependent antioxidant response. Thus, in summary, it is indicated that in vivo DMF and MMF show about the same the efficacy, in particular on the transcription factor Nrf2.

As mentioned above when taken orally DMF is hydrolyzed for example by the acidic ambience of the stomach or by esterases in the intestine to monomethyl fumarate (MMF). Thus, significant amounts of active agent are provided within a short time period. The above-indicated rate of hydrolysis was in principle expected to provide a significant level of MMF in the plasma within a short period of time. However, it seems that the MMF level still can be further improved to enable a faster onset of the intended pharmacological effect.

Further, it is reported that DMF has to be administered in high amounts and that the pharmaceutical active agent is supposed to show undesirable side effects such as flush and especially symptoms related to the gastrointestinal tract such as irritation of the stomach and diarrhoea.

Consequently, there is a need for new medicaments, which enable a fast pharmacological effect and/or which can be applied in appropriate doses and which preferably do not cause significant undesired side effects.

Hence, it was an object of the present invention to overcome the drawbacks of the above-mentioned substances.

It was an object to provide compounds suitable to be used as a medicament for the above-mentioned diseases, wherein preferably said compound shows advantageous pharmacokinetic properties.

In particular, compounds should be provided which in the intestine are hydrolyzed to MMF faster than DMF.

Further, the compounds should preferably not cause any undesirable side effects.

Additionally, it was an object of the present invention to provide compounds which can be used in the treatment of the early phase of phase of an autoimmune disease, in particular of multiple sclerosis.

### SUMMARY OF THE INVENTION

According to the present invention, the above objectives are achieved by specific compounds described herein by Formula (I) or (II). Said compounds can be used as a medicament preferably for the treatment and/or the prevention of systemic diseases, autoimmune diseases, inflammatory diseases, for example multiple sclerosis and psoriasis.

The compounds of the invention can be regarded as prodrugs of MMF. Generally, a prodrug can be regarded as a substance that is administered to a subject (preferably human) in a pharmacologically inactive or pharmacologically less than fully active form, and is subsequently converted in the body of the subject to an active drug, preferably through metabolic processes occurring in the body of the subject. In other words, a prodrug usually serves as a type of ‘precursor’ to the intended drug.

Thus, the subject of the present invention is a compound for use as a medicament according to the following Formula (I) or (II):

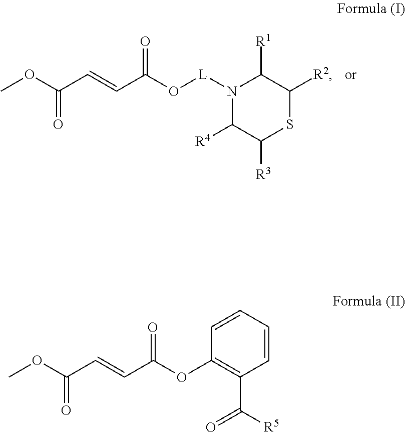

wherein R¹, R² R³, R⁴ and R⁵ are each independently an organic residue and L is an alkanediyl residue with 1 to 6 carbon atoms or alternatively 2 to 6 carbon atoms.

It was found that a compound according to Formula (I) or (II) of the present invention show excellent pharmaceutical and/or pharmacokinetic properties. In particular, the compounds show an excellent rate of hydrolysis to MMF in the intestine being faster than the one of DMF.

Further, the present invention relates to a compound according to Formula (I) or Formula (II) for use in the treatment and/or the prevention of systemic diseases, autoimmune diseases or inflammatory diseases, preferably for use in the treatment of multiple sclerosis or psoriasis, in particular multiple sclerosis.

Another subject is a pharmaceutical composition comprising the above-mentioned compound according to Formula (I) or (II).

Another subject of the present invention is the process of producing a compound according to the present invention by reacting monomethyl fumarate with the hydroxy group of a hydroxyl alkyl thiomorpholino derivative or with the hydroxy group of a salicylic acid derivative.

### DETAILED DESCRIPTION OF THE INVENTION

In the context of this invention, the compound of the present invention is represented by one of the Formula (I) or (II). Further, the compounds may refer to pharmaceutically acceptable salts, hydrates, solvates, polymorphs, stereoisomers and mixtures thereof.

In a particularly preferred embodiment of the present invention a single compound according to one of Formula (I) or (II) can be used as a medicament.

The same applies to the pharmaceutical composition comprising one of the compounds which are represented by Formula (I) or (II).

In a preferred embodiment of the invention in a compound according Formula (I) R¹, R², R³ and R⁴ are each independently hydrogen, methyl or halogen.

Halogen for example can be fluoride, chloride, bromide or iodide, preferably chloride or fluoride, in particular fluoride.

In a further preferred embodiment R¹, R², R³ and R⁴ are independently hydrogen or methyl.

In another preferred embodiment R¹, R², R³ and R⁴ can be hydrogen or methyl, in particular hydrogen. It is further preferred that R¹, R², R³ and R⁴ are the same residue, in particular hydrogen.

L is an alkanediyl residue with 1 to 6 carbon atoms.

Alkanediyl residues comprise linear and branched alkanediyl residues. Examples for alkanediyl residues are for example —CH₂—, —(CH₂)₂—, —CH(CH₃)—, —(CH₂)₃—, —CH₂CH(CH₃)—, —CH(CH₃)CH₂—, —CH(C₂H₅)—, —C(CH₃)₂—, —(CH₂)₄—, —(CH₂)₂CH(CH₃)—, —CH₂CH(CH₃)CH₂—, —CH(CH₃)(CH₂)₂—, —CH(C₂H₅)CH₂—, —CH₂CH(C₂H₅)—, —C(CH₃)₂CH₂—, —CH₂C(CH₃)₂—, —CH(CH₃)CH(CH₃)—, —CH(C₃H₇)—, —(CH₂)₅, —(CH₂)₃CH(CH₃), —(CH₂)₂CH(CH₃)CH₂—, —CH₂CHCH₃(CH₂)₂—, —CH₂C(CH₃)₂CH₂—, —(CH₂)₂C(CH₃)₂—, —(CH₂)₆—, —(CH₂)₄CH(CH₃)—, —(CH₂)₃CH(CH₃)CH₂—, —CH₂CHCH₃(CH₂)₃—, —(CH₂)₃C(CH₃)₂— and —(CH₂)₂C(CH₃)₂CH₂—.

In a preferred embodiment L is a linear alkanediyl residue with 1 to 6 carbon atoms, preferably with 2, 3 or 4 carbon atoms, more preferably with 2 or 4 carbon atoms, in particular with 2 carbons atoms.

In a preferred embodiment the inventive compound is represented by the following Formula (Ia)

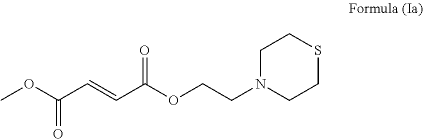

In Formula (Ia) L is —(CH₂)₂— and R¹, R², R³ and R⁴ are hydrogen.

In this regard it is noted that in the present invention the compound according to Formula (I) refers to a compound according to Formula (I) or its polymorphs, stereoisomers, solvates or hydrates, as well as pharmaceutically acceptable salts and mixtures thereof.

The compound according to Formula (I) can preferably comprise the pharmaceutically acceptable acid addition salts of the inventive compound. The acids which are used to prepare the pharmaceutically acceptable acid addition salts are preferably those which form non-toxic acid addition salts, i.e. salts containing pharmacologically acceptable anions, such as chloride, bromide, iodide, nitrate, sulfate, bisulfate, phosphate, acid phosphate, acetate, lactate, citrate, (D,L)- and L-tartrate, (D,L)- and L-malate, bitartrate, succinate, maleate, fumarate, gluconate, saccharate and benzoate. A preferred salt is the hydrochloride of a compound according to Formula (I), in particular of Formula (Ia).

A compound according to Formula (I) can preferably be synthesized via the following route:

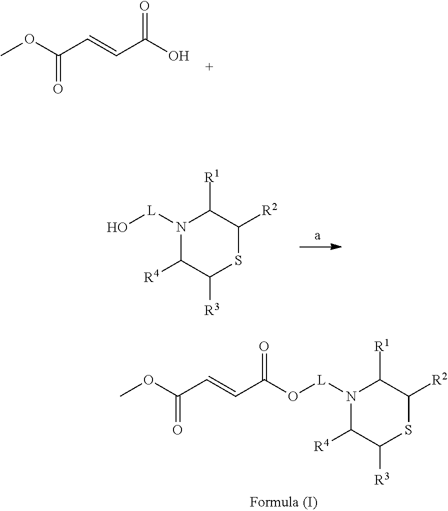

Preferably, in step a, MMF and the hydroxyl alkyl thiomorpholine derivative can be submitted to an esterification in an organic solvent in the presence of a coupling agent. A coupling agent is preferably a substance generally facilitating the formation of an ester or an amide. The coupling agent reacts with a carboxy group by forming a reactive intermediate which is subsequently further reacted with an alcohol or an amine to form the final product, i.e. an ester or an amide. Coupling agents are reported to be used in case that one or both of the educts further bear a group being labile in acidic or alkaline milieu, since the reaction is carried out under neutral conditions. Suitable coupling agents can be for example DCC (N,N′-dicyclohexylcarbodiimide), DIC (N,N′-diisopropylcarbodiimide), EDC (N-ethyl-N′-(3-methylaminopropyl)carbodiimide hydrochloride), CDI (carbonyldiimidazole), preferably EDC in combination with DMAP (4-(dimethylamino)pyridine).

A suitable organic solvent can for example be dichloromethane, chloroform, acetonitrile, dioxane, tetrahydrofuran and dimethyl formamide.

Alternatively, MMF can be preferably reacted with thionylchloride or oxalylchloride, preferably oxalychloride, to form the corresponding acid chloride. Subsequently the corresponding acid chloride can be submitted to a reaction with hydroxyl alkyl thiomorpholino derivative, preferably in an organic solvent, such as dioxane, tetrahydrofuran, chloroform or dichloromethane. Further, the reaction of the acid chloride with hydroxyl alkyl thiomorpholino derivative is preferably carried in the presence of an auxiliary alkaline compound. Suitable alkaline compounds are for example pyridine and amines, such triethylamine, and diisopropylethylamine preferably triethylamine.

In another embodiment of the invention R⁵ in Formula (II) is preferably OR^{5′} or NR^{5″}R^{5′″},

wherein R^{5′} is hydrogen, alkyl with 1 to 6 carbon atoms or cyclic alkyl with 3 to 6 carbon atoms, or

wherein R^{5″} and R^{5′″} independently are hydrogen, alkyl with 1 to 6 carbon atoms or cyclic alkyl with 3 to 6 carbon atoms.

Alkyl with 1 to 6 carbon atoms can for example include methyl, ethyl, propyl, isopropyl, butyl, isobutyl, tert.butyl, pentyl, sec.-pentyl, and hexyl.

Cyclic alkyl with 3 to 6 carbon atoms can for example include cyclopropyl, cyclobutyl, cyclopentyl and cyclohexyl.

In a preferred embodiment R⁵ is OR^{5′}, wherein R^{5′} can be hydrogen, alkyl with 1 to 6 carbon atoms or cyclic alkyl with 3 to 6 carbon atoms, preferably alkyl with 1 to 6 carbon atoms, more preferably alkyl with 1 to 3 carbon atoms, in particular ethyl.

In a preferred embodiment of the inventive compound is represented by the following Formula (IIa)

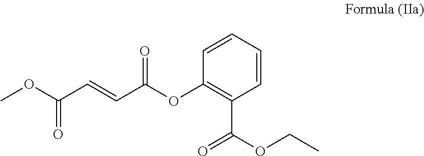

In Formula (IIa) R⁵ is OEt.

In an alternatively preferred embodiment R⁵ is NR^{5″}R^{5′″}, wherein R^{5″} and R^{5′″} independently can be hydrogen, alkyl with 1 to 6 carbon atoms or cyclic alkyl with 3 to 6 carbon atoms.

It is further preferred that one of R^{5″} and R^{5′″} is hydrogen. It is further preferred that the other residue can be alkyl with 1 to 6 carbon atoms, preferably alkyl with 1 to 3 carbon atoms.

It is alternatively preferred that R^{5″} and R^{5′″} are identical. It is further preferred that they are hydrogen or alkyl with 1 to 3 carbon atoms.

In this regard it is noted that in the present invention the compound according to Formula (II) refers to a compound according to Formula (II) or its polymorphs, stereoisomers, solvates or hydrates, as well as pharmaceutically acceptable salts and mixtures thereof.

A compound according to Formula (II) can preferably be synthesized via the following route:

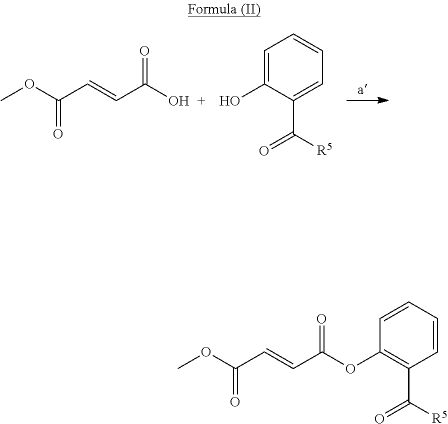

Generally the conditions for step a′ correspond to the ones of step a as mentioned above. MMF and the hydroxyl group of the salicylic acid derivative can be submitted to an esterification in an organic solvent in the presence of a coupling agent.

Further, also as mentioned above under step a, MMF can be preferably reacted with thionyl chloride or oxalyl chloride, preferably oxalyl chloride, to form the corresponding acid chloride and the reaction is conducted under the same conditions as above.

The above compounds according to Formulae (I) and (II) show excellent pharmacokinetic properties.

Within one hour the inventive compounds show a hydrolysis into MMF and remaining organic residue wherein the hydrolysis is faster than the one of DMF. As a result a greater amount of MMF is released within the one hour and thus the compounds can be referred to as compounds (prodrugs of MMF) with an enhanced release of MMF. Additionally the remaining organic residue is not expected to harm the patient's organism.

A further subject of the invention is the inventive compound for use in the treatment and/or prevention of systemic diseases, autoimmune diseases or inflammatory diseases.

Systemic diseases do not just affect single organs. Instead these diseases are known to affect a number of organs and tissues or even the body as a whole.

People having an autoimmune disease usually suffer from their immune system mistakenly attacking their own cells of their organism and thus incorrectly responding to substances normally present in the body.

An inflammation can be defined as the response of the body to the occurrence of harmful stimuli which can result in pain, heat, redness, swelling and loss of function of the affected organ.

It is possible that some of the above-mentioned diseases cannot be allocated in one single group of the above-mentioned groups, since they show the symptoms of more than one of them.

In a further preferred embodiment the inventive compounds is for use in the treatment of multiple sclerosis and psoriasis, preferably multiple sclerosis. The compounds of the present invention can e.g. be used in the treatment of the following types of multiple sclerosis, relapsing-remitting, primary-progressive, secondary-progressive, and progressive-relapsing. In preferred embodiment the compounds of the present invention are used in the treatment of relapsing-remitting multiple sclerosis.

Further, the present invention also provides a pharmaceutical composition comprising the compound according to the present invention, i.e. a pharmaceutical composition comprising a prodrug of MMF according to Formula (I) or (II) and optionally pharmaceutical excipients.

In a preferred embodiment the pharmaceutical composition comprises
(i) 0.01 to 10 mmol, more preferably 0.05 to 5 mmol, still more preferably 0.25 to 3.5 mmol and particularly preferred 0.5 to 2.5 mmol of a compound according to the present invention;
(ii) pharmaceutical excipient(s).

In a further preferred embodiment the present composition can comprise one or more further excipients, preferably pharmaceutical excipients as described in the European Pharmacopoeia (Ph.Eur.) and/or in the US Pharmacopoeia (USP).

Examples of pharmaceutical excipients are carriers, binders, fillers, disintegrants, wicking agents, glidants and/or lubricants.

In a preferred embodiment the excipients are chosen such that the resulting formulation is a gastric juice-resistant formulation. In a preferred embodiment the formulation of the present invention does not show significant drug release under acidic conditions. In particular, the in-vitro drug release after 2 hours is less than 10%, preferably 0 to 9.9%, more preferably 0 to 5%, still more preferably 0.001 to 3%, measured according to USP, Apparatus II, paddle, 0.1N HCl, 37° C., 50 rpm.

The pharmaceutical composition can be in a form suitable for oral administration, preferably in the form of a tablet or capsule, in particular in form of a tablet.

It is further preferred that the tablet is coated with a film coating. Alternatively, the capsule could also be coated.

In the present invention, the following three types of film coatings are possible:
film coating without affecting the release of the active ingredient,
gastric juice-resistant film coatings,
retard film coatings.

Generally, film coatings can be prepared by using film-forming agents such as waxes, cellulose derivatives, poly(meth)acrylate, polyvinylpyrrolidone, polyvinyl acetate phthalate, and/or shellac or natural rubbers such as carrageenan.

It is preferred that the present tablet is coated with a gastric juice-resistant film coating. Alternatively, a capsule comprising a gastric juice-resistant film coating can be used.

The gastric juice-resistant film coating preferably is a film coating being stable in the pH range of about 0.7 to 3.0, which is supposed to be the pH-value of human gastric juice found in the stomach. However, in an environment with a pH value of 5 to 9, which is supposed to be present in the (small) intestine of the human body, the gastric juice-resistant film coating preferably dissolves and the drug can be released.

The gastric juice-resistant film coating (often also referred to as enteric coating) can comprise film-forming agents being for example fats, fatty acids, waxes, alginates, shellac, polyvinyl acetate phthalate, cellulose derivatives such as carboxy methyl ethyl cellulose, cellulose acetate succinate, cellulose acetate phthalate, hydroxypropyl methyl cellulose phthalate, hydroxypropyl methyl cellulose acetate succinate, cellulose acetate trimellitate, and meth(acrylic)acid copolymers such as methyl acrylate-methacrylic acid copolymers, methyl methacrylate-methacrylic acid copolymers, Eudragits (for example Eudragit® L30D, Eudragrt® L, Eudragit® S).

The coating is preferably free of active ingredient. It is further preferred that the thickness of the coating is usually 10 μm to 2 mm, preferably from 50 to 500 μm.

The preferred coating may comprise a film-forming agent and one or more of the following: lubricant, surfactant, glidant, pigment and water.

The preferred coating according to an embodiment of the present invention can comprise, along with the film-forming agent, e.g. stearic acid as lubricant for plasticizing and dissolving the polymer, sodium lauryl sulfate as a surfactant for wetting and dispersing, talc as glidant, iron oxide yellow and/or titanium oxide as pigment(s) and optionally purified water.

In a preferred embodiment the pharmaceutical composition can be administered one to three times a day, preferably once or twice a day, more preferably once a day.

The invention is illustrated by the following examples.

### EXAMPLES

### Example 1

### Synthesis of (E)-But-2-enedioic acid 2-(thiomorpholin-4-yl)-ethyl ester methyl ester hydrochloride

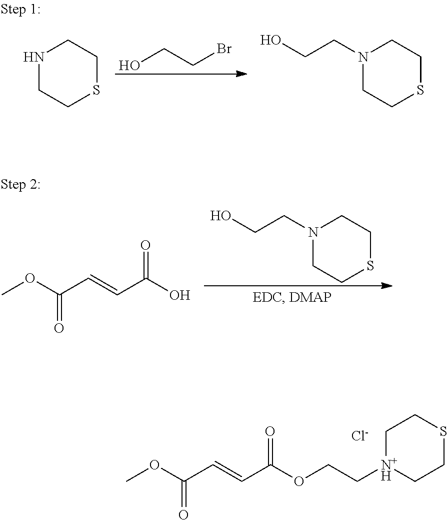

### Step 1: Preparation of 2-Thiomorpholin-4-yl ethanol

To a solution of thiomorpholine (3 g; 29.1 mmol) in acetonitrile (40 mL) were added potassium carbonate (12 g, 87.2 mmol) and 2-bromoethanol (6.18 ml, 87.2 mmol) and the suspension stirred under reflux for 16 h. After complete conversion dichloromethane (150 mL) was added to this suspension, the solid was removed by filtration and the filtrate was concentrated under reduced pressure. The colorless oil was directly used for step 2 without purification.

### Step 2

To a suspension of monomethyl fumarate (1.5 g; 11.5 mmol) in dichloromethane (30 mL) were added at 0° C. were added 1-Ethyl-3-(3-dimethylaminopropyl)carbodiimid hydrochloride (EDC; 2.65 g, 13.8 mmol), 2-thiomorpholin-4-yl ethanol (as obtained from step 1; 2.43 g) and DMAP (0.1 g; 1.2 mmol) and the mixture was stirred overnight at room temperature. It was diluted with dichloromethane (50 ml), washed with water (2×70 mL), dried over sodium sulfate and concentrated under reduced pressure. The brown residue was taken up in diethylether (40 mL), filtered and to the yellow filtrate was added HCl (3 M in n-butanol; 6 mL). The immediately formed precipitate was filtered off, washed with diethylether and tetrahydrofuran and finally recrystallized from chloroform.

Yield: 700 mg

Chemical purity (HPLC, area-% at λ=226 nm): 99.3%

### Example 2

### (E)-But-2-enedioic acid -2-(ethoxycarbonyl-phenyl)ester methyl ester

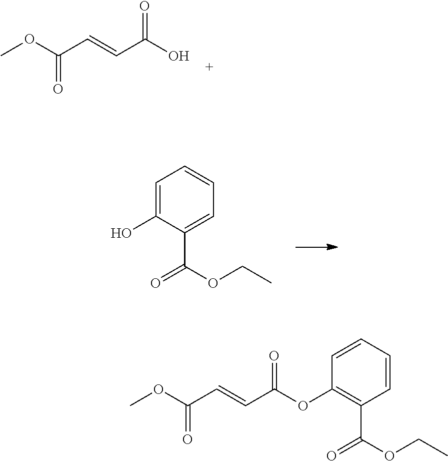

To a suspension of monomethyl fumarate (1.5 g 11.5 mmol) in dichloromethane (30 mL) were added at 0° C. 1-Ethyl-3-(3-dimethylaminopropyl)carbodiimide hydrochloride (EDC; 2.65 g (13.8 mmol), ethyl salicylate (1.70 ml, 11.5 mmol) and DMAP (0.1 g; 1.2 mmol) and the mixture was stirred overnight at room temperature. Additional dichloromethane (100 mL) was added and the solution was washed with water (2×50 mL), dried over sodium sulfate and concentrated under reduced pressure. The oily residue was subjected to silica gel chromatography (eluent: n-hexane/methyl tert.-butylether (4:1-3:1)) to obtain pure (E)-But-2-enedioic acid -2-(ethoxycarbonyl-phenyl)ester methyl ester.

Yield: 410 mg

Chemical purity (HPLC, area-% at λ=226 nm): 99.0%

### Example 3

### (E)-But-2-enedioic acid 2-dimethylcarbamoyl-phenyl ester methyl ester

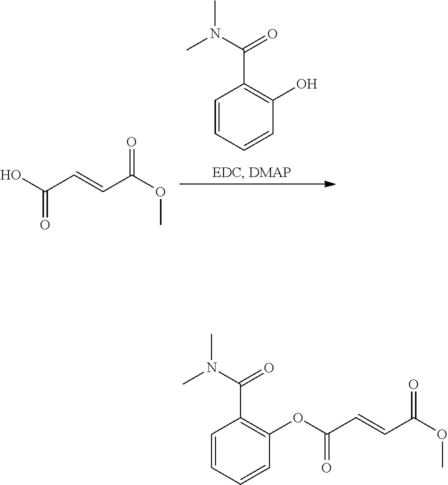

To a stirred suspension of monomethyl fumarate (0.8 g; 6.1 mmol) in dry dichloromethane (16 mL) was added 1-Ethyl-3-(3-dimethylaminopropyl)carbodiimide hydrochloride (EDC; 1.41 g; 7.4 mmol) at 0° C. and the resulting redbrown mixture was stirred for 5 min after 2-hydroxy-N,N-dimethylbenzamide (0.97 g; 5.8 mmol) and DMAP (80 mg; 0.6 mmol) were added successively. The mixture was allowed to warm to room temperature and stirring was continued overnight (16 h). The solution was diluted with additional dichloromethane (50 mL), washed with water (2×40 mL), dried over sodium sulfate and concentrated under reduced pressure. The crude product was subjected to silicagel chromatography (eluent: n-hexane/ethyl acetate 3:1) to obtain pure (E)-But-2-enedioic acid 2-dimethylcarbamoyl-phenyl ester methyl ester.

Yield: 290 mg.

### Example 4

### l Investigation and Comparison of the Kinetics of MMF-Release of the Different Compounds of the Present Invention and DMF During Incubation in Intestinal Fluid of the Minipig

1. Materials

1.1. Test Compounds

Compounds of the present invention were synthesized as described above.

1.2. Intestinal Fluid

Intestinal fluid samples were prepared at CiToxLAB Scantox A/S. The samples were taken from 1 female Gottingen SPF minipig from CiToxLAB Scantox A/S standard stock, originally obtained from Ellegaard Gottingen Minipigs A/S, DK-4261 Dalmose, Denmark. The minipig was 10 months old and the body weight was 21 kg. The minipig was identified by an individual number tagged to the pinna of one ear (animal number is documented in the raw data).

The minipig was fasted for approximately 28 hours before sampling of intestinal fluid. On the day of sampling, the minipig was weighed and anaesthetised by an intramuscular injection in the neck or in the left hind leg (about 0.3 ml per kg body weight) of a mixture of Zoletil 50 Vet., Virbac, France (125 mg tiletamine and 125 mg zolazepam), Rompun Vet., Bayer, Germany (20 mg xylazine/ml, 6.5 ml), Ketaminol Vet., Veterinaria AG, Switzerland (100 mg ketamine/ml, 1.5 ml) and Methadon DAK, Nycomed Danmark, Denmark (10 mg methadon/ml, 2.5 ml).

Intestinal fluid was obtained by flushing one jejunal segment, measuring 30.2 cm, with saline. Intestinal fluid together with saline used for flushing was placed in centrifuge tubes. All samples were frozen at −70° C. and shipped on dry ice to the Sponsor for further use.

2. Analytical Methods

2.1. Quantification of MMF by LC-MS

2.1.1. Analytical Instrument
Instrument: Acquity UPLC system coupled with a TQ detector (triple quadruple mass spectrometer)

UPLC Method:

Column: Phenomenex Kinetex C18, 100A, 2.6 μm (150×4.6 mm)

flow: 0.4 ml/min

split: appr. 100 μl/min to MS

temperature 30° C.

solvet system (isocratic):
Solvent A 25% water with 0.1% acetic acid
Solvent B 75% methanol with 0.1% acetic acid

stoptime: 6 min

autosampler temperature: 8° C.

injection volume: 4 μl

retention time: MMF: 4.3 min
MEF: 4.7 min

Mass Spectrometry

software: Masslynx 4.1

detection mode: electrospray/negative ions (ESP−)

capillary voltage: 2.3 kV

source temperature: 100° C.

desolvation temperature: 450° C.

cone voltage: 18 V

desolvation gas: N₂, 650 L/h

cone gas: N₂, 20 L/h

collision gas: argon, appr. 3.3*10⁻³ mbar

collision energy: 11 eV

MRM [m/z]: 128.94>85.03 Monomethylfumarate dwell: 200 msec
142.99>99.06 Monoethylfumarate (ISTD) dwell: 200 msec

2.1.2. Stock and Calibration Solutions

Stock (SS), working (WS) and calibration solutions of the analyte monomethyl fumarate (MMF) and the internal standard (ISTD) monomethyl fumarate (MEF) were prepared as described below.
SS_{MMF}: In a 10 ml volumetric flask, 6.5 mg MMF (Batch: MKRJ0642V/Aldrich) were dissolved in methanol and made up to volume (c=650 μg/ml)
SS_{ISTD}: In a 100 ml volumetric flask, 10 mg MEF (Batch: STBC5219V/Aldrich) were dissolved in methanol and made up to volume (c=100 μg/ml)
   WS_{ISID}: 100 μl SSISTD were transferred into a 10 ml volumetric flask and made up to volume with acetonitrile (c=1,000 ng/ml);

Calibration solutions were prepared by serial dilution of SS_{MMF}; diluted small intestinal fluid (diluted by 1/20 v/v with 50 mM KH₂PO₄, pH 6.8; (dil IF) was used as matrix. The dilution scheme is given below:

| calibration | | | Concentration | |
|---|---|---|---|---|
| solution | Preparation | | [ng/ml] | [μM] |
| cal6500 | 8 μl SS_{MMF} | +792 μl dil IF | 6,500 | 50 |
| cal3250 | 50 μl cal6500 | +50 μl dil IF | 3250 | 25 |
| cal650 | 20 μl cal6500 | +180 μl dil IF | 650 | 5.0 |
| cal325 | 50 μl cal650 | +50 μl dil IF | 325 | 2.5 |
| cal65 | 10 μl cal650 | +90 μl dil IF | 65 | 0.5 |

2.1.3. Sample Preparation

50 μl sample (calibration solution or sample of an incubation experiment with MMF prodrugs) was mixed with 50 μl WS_{ISTD}, 20 μl formic acid and 100 μl acetonitrile. This mixture was vortexed for 15 sec and centrifuged (13,000 rpm, 3 min). Thereafter, 4 μl of the supernatant were subjected to LC-MS analysis.

2.2. Incubation Experiments with DMF (Reference) and Compounds of the Invention

2.2.1. Stock Solutions

Stock solutions were prepared in DMSO. Concentrations in stock solutions were 5.00, 2.50 and 1.67 mmol for compounds with one, two and three molar MMF equivalents.

| | | Sample | | | |
|---|---|---|---|---|---|
| | | weight | | Concentration | |
| Compound | MW | [mg] | dissolved in | [mg/ml] | [mmol] |
| DMF | 144.13 | 7.21 | 10 ml DMSO | 0.721 | 5.00 |
| Example 1 | 295.79 | 7.39 | 5 ml DMSO | 1.478 | 5.00 |
| Example 2 | 278.26 | 6.96 | 5 ml DMSO | 1.392 | 5.00 |

2.2.2. Incubation Experiment

In a HPLC glass vial, 8 μl of stock solution were mixed with 792 μl dil IF and the mixture was stirred (250 rpm) in a water bath (T=37° C.).

Immediately after mixing as well as at t=15 min, 30 min, 60 min, 90 min and 120 min, 50 μl were withdrawn and prepared for LC-MS analysis as described in chapter. 2.1.3.

Incubations were continued and in case the result of analysis of the 120 min indicated the presence of remaining intact MMF prodrug, additional samples were taken (t=360 or 420 min and at 1,260 or 1,320 min) and analysed.

3. Results

3.1. Calibration of the Analytical Method

Each calibration solution was analysed two-fold. The second analysis was carried out approx. 18 h after storage of the sample in the autosampler, which was cooled to 8° C. The results demonstrate that the ratio of peak area remains essentially unchanged between the first and the second analysis.

The concentration/peak area ratio data pairs were subjected to regression analysis with 1/x weighting and the resulting calibration equation was used to quantify the MMF content in incubation samples.

| | nominal | | | | |
|---|---|---|---|---|---|
| calibration | concentration | | area/ | | |
| standard | [ng/ml] | Analysis | area (ISTD) | mean | RSD |
| cal6500 | 6,500 | 1^{st} analysis | 3.569 | 3.567 | 0.07 |
| | | 2^{nd} analysis | 3.564 | | |
| cal3250 | 3,250 | 1^{st} analysis | 1.710 | 1.681 | 1.73 |
| | | 2^{nd} analysis | 1.652 | | |
| cal650 | 650 | 1^{st} analysis | 0.348 | 0.347 | 0.29 |
| | | 2^{nd} analysis | 0.346 | | |
| cal325 | 325 | 1^{st} analysis | 0.174 | 0.169 | 2.96 |
| | | 2^{nd} analysis | 0.164 | | |
| cal65 | 65 | 1^{st} analysis | 0.036 | 0.035 | 2.86 |
| | | 2^{nd} analysis | 0.034 | | |
| cal0 | 0 | 1^{st} analysis | 0.000 | 0.000 | 0.00 |
| | | 2^{nd} analysis | 0.000 | | |

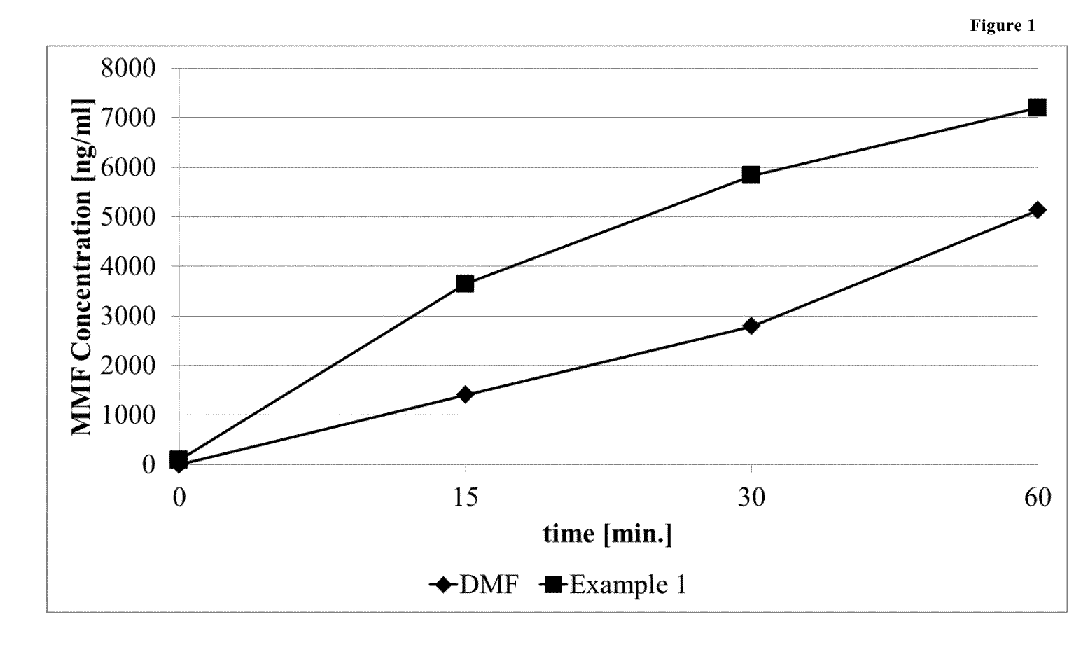

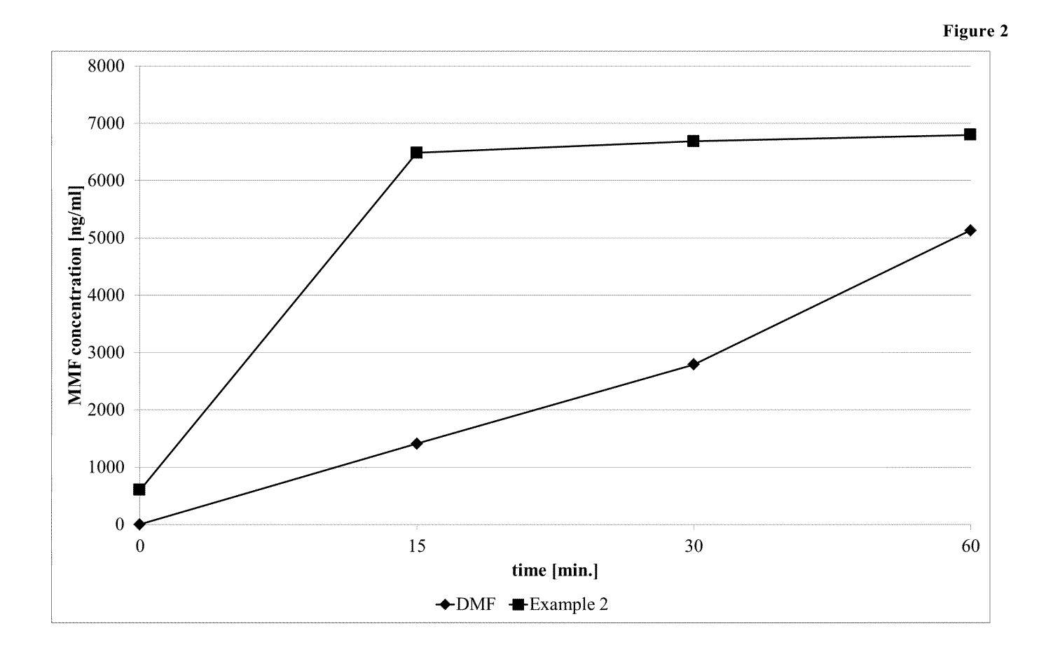

As can be seen from FIGS. 1 and 2 the inventive compounds according to Formulae (I) and (II) show a faster hydrolysis to MMF than DMF.

### Example 5

### Assessment and Comparison of the Efficacy of Compounds of the Invention and DMF (Reference) in MOG₃₅₋₅₅-Induced Experimental Autoimmune Encephalomyelitis (EAE) in C57BL/6 Mice

Test System:

male C57BL/6 mice, 12 weeks old; 10 animals per treatment group;

Induction of EAE:

Day 1—subcutaneous injection of MOG₃₅₋₅₅, suspended in complete Freund's adjuvans and intraperitoneal injection of pertussis toxin.

Day 3—intraperitoneal injection of pertussis toxin.

Treatment:

Dimethylfumarate and test substances or vehicle only were administered via oral and intravenous route.

For oral administration, test substances were dissolved or suspended in 0.5% hydroxyethylcellulose (dissolved in 50 mM potassium dihydrogenphosphate, pH 5.0). Drug concentration in dose formulations: 11.54 mM;

Dose volume: 10 ml/kg body weight;

Start of treatment: Day 3

For intravenous administration, stock solutions were prepared in DMSO and serially diluted with phosphate buffered saline.

Observations (Clinical Score and Body Weight):

Observations were recorded on day 2, 4, 6, 8, 10, 12, 14 and 16.

Clinical score: grade 0-10; 0 (=no impairments), 1 (normal movement; limp tail: proximal 2/3 of the tail is limp and droopy), 2 (normal movement; whole tail is limp; 3 (wobbly walk; absent righting reflex), 4 (gait ataxia), 5 (milad paraparesis), 6 (moderate paraparesis), 7 (severe paraparesis or paraplegia), 8 (tetraparesis), 9 (moribund), 10 (death).

Results:

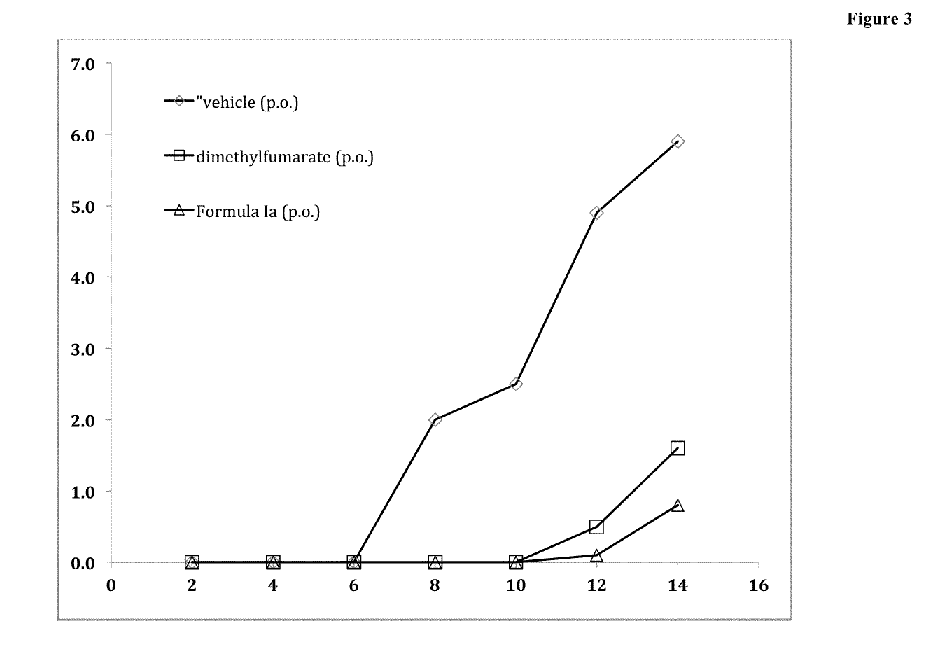

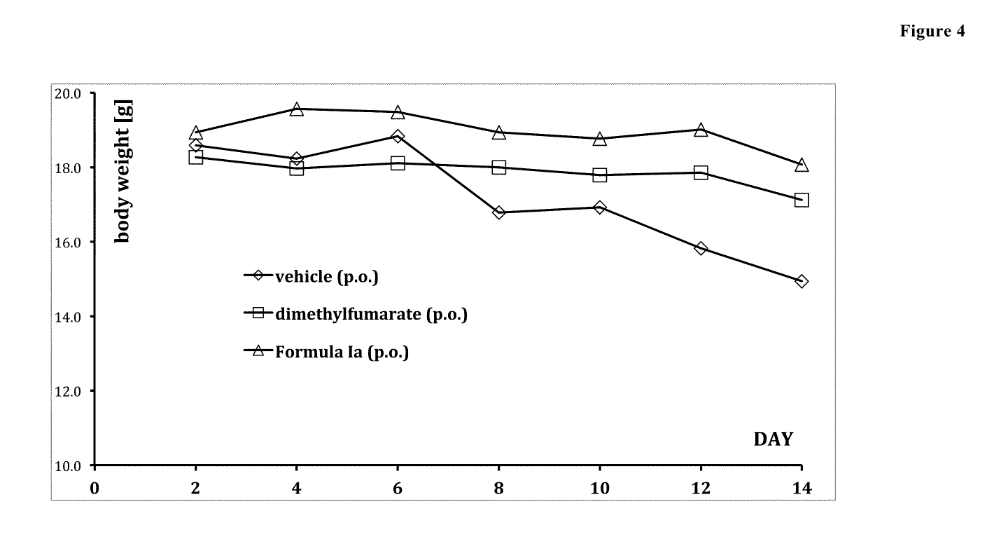

For test substance Ia it is shown in FIG. 3 that clinical score is reduced with reference to DMF, and in FIG. 4 that the body weight is substantially maintained. The reduction of the clinical score and/or the maintenance of the body weight can indicate the effectiveness of the treatment.

1. Compound for use as a medicament according to Formula (I) or (II) 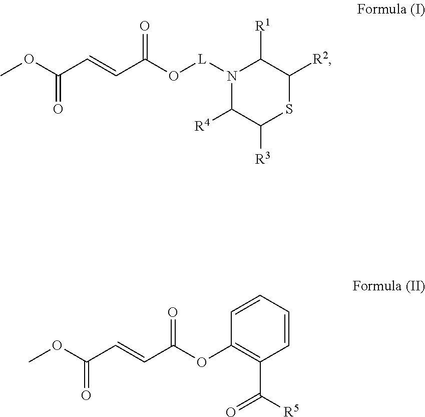
wherein R¹, R² R³, R⁴ and R⁵ are each independently an organic residue and L is an alkanediyl residue with 1 to 6 carbon atoms.

2. Compound according to claim 1, wherein R¹, R² R³ and R⁴ in Formula (I) are each independently hydrogen, methyl or halogen.

3. Compound according to claim 1 or 2, wherein R¹, R² R³ and R⁴ are hydrogen.

4. Compound according any one of claims 1 to 3, wherein L is a linear alkanediyl residue.

5. Compound according to any one of claims 1 to 4 represented by Formula (Ia)

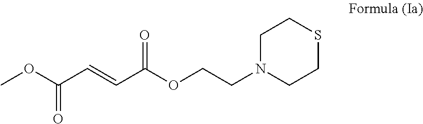 [SMILES: COC(=O)/C=C/C(=O)OCCN1CCSCC1]

6. Compound according to claim 1, wherein in Formula (II) R⁵ is OR^{5′} or NR^{5|}R^{5′″},
wherein R^{5′} is hydrogen, alkyl with 1 to 6 carbon atoms or cyclic alkyl with 3 to 6 carbon atoms, or
wherein R5″ and R^{5′″} independently are hydrogen, alkyl with 1 to 6 carbon atoms or cyclic alkyl with 3 to 6 carbon atoms.

7. Compound according to claim 1 or 6, wherein R⁵ is OR^{5′}, wherein R^{5′} is alkyl with 1 to 6 carbon atoms.

8. Compound according to any of claims 1, 6 and 7 represented by Formula (IIa)

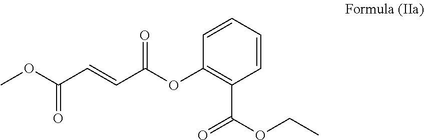 [SMILES: CCOC(=O)c1ccccc1OC(=O)/C=C/C(=O)OC]

9. Compound according to any one of claims 1 to 8 for use as a medicament.

10. Compound according to any one of claims 1 to 8 for use in the treatment of systemic diseases, autoimmune diseases or inflammatory diseases, preferably for the use in the treatment of multiple sclerosis or psoriasis.

11. Pharmaceutical composition comprising a compound according to any one of claims 1 to 8.

12. Pharmaceutical composition according to claim 11, comprising
(i) 0.01 to 10 mmol of a compound according to any one of claims 1 to 8, and
(ii) optionally pharmaceutical excipients.

13. Pharmaceutical composition according to claim 11 or 12, wherein the composition is a solid oral dosage form.

14. Pharmaceutical composition according to any of claims 11 to 13, wherein the in-vitro drug release after 2 hours is less than 10%, measured according to USP, Apparatus II, paddle, 0.1 N HCl, 37° C., 50 rpm.
